SEQUENCE LISTING (6 sequences)

SEQ ID NO: 1 - DNA, 23 bp, Artificial Sequence
  GAPDH primer (sense)
gtgaaccatgagaagtatgacaa

SEQ ID NO: 2 - DNA, 20 bp, Artificial Sequence
  GAPDH primer (antisense)
catgagtccttccacgatac

SEQ ID NO: 3 - DNA, 20 bp, Artificial Sequence
  MMP-1 primer (sense)
ctgaaggtgatgaagcagcc

SEQ ID NO: 4 - DNA, 20 bp, Artificial Sequence
  MMP-1 primer (antisense)
agtccaagagaatggccgag

SEQ ID NO: 5 - DNA, 20 bp, Artificial Sequence
  MMP-3 primer (sense)
ctcacagacctgactcggtt

SEQ ID NO: 6 - DNA, 20 bp, Artificial Sequence
  MMP-3 primer (antisense)
cacgcctgaaggaagagatg

(19) United States
(12) Patent Application Publication
(10) Pub. No.: US 2021/0070866 A1
(43) Pub. Date: Mar. 11, 2021

(54) Pharmaceutical Composition For Preventing Or Treating Tissue Adhesion Comprising Integrin a2ß1 Inhibitors

(71) Applicant: KOREA UNIVERSITY RESEARCH AND BUSINESS FOUNDATION, Seoul (KR)

(72) Inventor: Hong Joo MOON, Seoul (KR)

(73) Assignee: KOREA UNIVERSITY RESEARCH AND BUSINESS FOUNDATION, Seoul (KR)

(21) Appl. No.: 16/986,824

(22) Filed: Aug. 6, 2020

(30) Foreign Application Priority Data
Aug. 8, 2019 (KR) 10-2019-0096865

(51) Int. Cl. C07K 16/28; A61K 31/18; A61K 31/415; A61K 31/4155; A61K 38/17; A61K 9/06; A61P 41/00

(52) U.S. Cl. CPC C07K 16/2842; A61K 31/18; A61K 31/415; A61P 41/00; A61K 38/1703; A61K 9/06; A61K 31/4155

(57) ABSTRACT

Disclosed herein are a pharmaceutical composition comprising an integrin α2β1 inhibitor as an active ingredient for prevention or treatment of tissue adhesion, a method for screening a material prophylactic of or therapeutic for tissue adhesion, an anti-adhesion composition comprising the pharmaceutical composition for prevention or treatment of tissue adhesion as an active ingredient, and a method for prevention or treatment of tissue adhesion. The present disclosure can be advantageously used for preventing or treating tissue adhesion.

### FIELD

The present disclosure relates to a pharmaceutical composition comprising an integrin α2β1 inhibitor as an active ingredient for prevention or treatment of tissue adhesion.

BACKGROUND

Among major factors that cause postsurgical complications are dural traction by peridural adhesion and direct neural compression due to excessive fibrosis. Because peridural adhesion and fibrosis stem from cell-cell and cell-matrix interactions during inflammation, various approaches have been utilized to prevent adhesion by inhibiting specific components of the inflammatory process. Treatments with mitomycin C, rosuvastatin, and various synthetic polymeric materials alone or in combination have been developed as alternatives to autologous fat grafts, but with effects thereof remaining unsatisfactory to date.

Adhesion in epidural tissue is caused by the propagation of fibroblasts and fibroblast-producing collagen fibers while the fibroblasts originate from the injured sacrospinalis, intervertebral disc fibers, fiber ring, and posterior longitudinal ligament.

Because fibroblast adhesion and cellular responses associated therewith contribute to various aspects of wound healing, many studies have been conducted to clarify the adhesion mechanism of fibroblasts in these tissues. Adhesion mechanisms are known to include i) enhanced collagen synthesis by fibroblasts that have migrated from paraspinal muscles, ii) inflammation triggered by intervertebral disc fibroblasts, and iii) activated IL-6 expression due to angiopoietin-like protein 2 binding in ligamentum flavum fibroblasts. In addition, because wound healing in epidural tissue closely correlates with the reaction to inflammation in dura mater cells, various studies have been conducted to investigate this relationship. As a result, studies using rabbits and mini-pigs revealed that most cultured dura mater cells are fibroblasts. However, results of a recently established human primary culture model differ from those of animal-based studies. Human dura mater cells (hDMCs) do not rely on collagen coating in culture and cell growth and do not exhibit significant inhibition of proliferation with the mitosis inhibitor mitomycin-C, unlike the results of animal experiments. Understanding the mechanism through which hDMCs adhere to components of the extracellular matrix (ECM) during inflammation mimicking postsurgical conditions may help prevent peridural adhesion.

Accordingly, the present inventors aimed to determine which components of ECM are major adhesive substrates to hDMCs, which transmembrane molecules in hDMCs have a major role in the adhesion process, and which secreted matrix metalloproteinases (MMP) linked with ECM remodeling could be altered after exposure to inflammation using primary hDMC cultures.

### SUMMARY

### Technical Problem

A purpose of the present disclosure is to provide a pharmaceutical composition comprising an integrin α2β1 inhibitor as an active ingredient for prevention or treatment of tissue adhesion.

Another purpose of the present disclosure is to provide a method for screening a material preventive of or therapeutic for tissue adhesion.

A further purpose of the present disclosure is to provide an anti-adhesion composition comprising the pharmaceutical composition for prevention or treatment of tissue adhesion and a biocompatible hydrogel.

A still further purpose of the present disclosure is to provide a method for prevention or treatment of tissue adhesion, the method comprising a step of administering the pharmaceutical composition to a subject.

### Solution to Problem

According to an aspect thereof, the present disclosure provides a pharmaceutical composition comprising an integrin α2β1 inhibitor as an active ingredient for prevention or treatment of tissue adhesion.

As used herein, “tissue adhesion” refers to a phenomenon in which neighboring and normally separate tissues adhere to each other as a result of the excessive generation of fibrous tissues or hemorrhaging and blood coagulation during a wound healing process after inflammation, scarring, abrasion, surgical cuts, or the like.

Integrin α2β1, which is a member of the 12 integrin family adhesion receptors having beta 1 subunit in common, is a receptor for laminin, collagen, collagen C-propeptide, fibronectin, and E-cadherin. Integrin α2β1 recognizes the proline-hydroxylated sequence G-F-P-G-E-Rfmf in collagen. Integrin α2β1 is responsible for adhesion of platelets and other cells to collagens, modulation of collagen and collagenase gene expression, and generation and organization of newly synthesized extracellular matrix.

In the pharmaceutical composition, an integrin α2β1 inhibitor is used as an active ingredient. The term “integrin α2β1 inhibitor”, as used herein, refers to a substance for downregulating the expression of integrin α2β1 protein or gene and for preventing or interfering with binding of extracellular matrix components (e.g., collagen) to integrin α2β1 to decrease the binding affinity.

In an embodiment of the present disclosure, as long as it downregulates an expression level of integrin α2β1 protein or gene, any integrin α2β1 inhibitor may be used. Particularly, the inhibitor may be an antibody, an aptamer, a natural extract, or a compound. More particularly, examples of the inhibitor include an anti-integrin α2β1 antibody or an antigen binding fragment thereof, BTT-3016 {sodium salt of 4-({[3-(4-fluorophenyl)phenyl]sulfonyl}amino)phenyl phenyl ketone}, BTT-3033 {1-(4-Fluorophenyl)-N-methyl-N-[4[[(phenylamino)carbonyl]amino]phenyl]-1H-pyrazole-4-sulfonamide}, BTT-3034 {N-[4-(methyl{[5-(1-methylpyrazol-5-yl)(2-thienyl)]sulfonyl}amino)phenyl]phenylamino)carboxamide}, and rhodocetin (C-type lectin protein found in Malayan pit viper (Calloselasma rhodostoma) venom), but are not limited thereto.

As used herein, the term “antibody” is intended to encompass not only a whole antibody form, but also an antigen-binding fragment thereof. A whole antibody includes two full length light chain and two full length heavy chains where each light chain is linked to the heavy chain by disulfide bonds. The heavy chain constant region is divided into isotypes of gamma (γ), mu (μ), alpha (α), delta (δ), and epsilon (ε) types, which are further subtyped into gamma1 (γ1), gamma2 (γ2), gamma3 (γ3), gamma4 (γ4), alpha 1 (α1), and alpha 2 (α2). The light chain constant region is divided into kappa (κ) and lambda (λ) types.

As used herein, the term “antigen-binding fragments” refers to a fragment retaining the function of binding to an antigen and includes Fab, F(ab′), F(ab′)2, and Fv. Of them, Fab (fragment antigen binding) is composed of one constant and one variable domain of each of the heavy and the light chain, the constant domain of the heavy chain being the first constant domain (CH1), and thus contains one antigen-binding site. Fab′ is different from Fab in that the former comprises a hinge region including at least one cysteine residue at the C-terminal of the CH1 domain of a heavy chain. F(ab′)2 is produced by a disulfide bond formation between cysteine residues in the hinge region of Fab′. Fv is an antibody fragment composed only of variable regions of a heavy and a light chain, which may be produced by a recombinant technology disclosed in the art. In Fv (two-chain Fv), variable regions of a light and heavy chain are linked by a non-covalent bond, and in a single chain Fv, variable regions of a light and heavy chain are linked by a covalent bond through a peptide linker or it may form a dimer structure like a two chain FV through a direct linkage at the C-terminal. These antibody fragments can be obtained through a proteinase treatment (for example, a whole antibody may be treated with a papain to obtain Fab fragments or with pepsin to obtain F(ab′)2 fragment) or preferably constructed using a recombinant DNA technology.

As used herein, the term “antibody or an antigen binding fragment thereof” encompasses not only full-length or intact polyclonal or monoclonal antibodies, but also antigen-binding fragments thereof (for example, Fab, Fab′, F(ab′)₂, Fab3, Fv and variants thereof), fusion proteins comprising one or more antibody portions, humanized antibodies, chimeric antibodies, minibodies, diabodies, triabodies, tetrabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies and covalently modified antibodies. Further examples of modified antibodies and antigen binding fragments thereof include nanobodies, AlbudAbs, DARTs (dual affinity re-targeting), BiTEs (bispecific T-cell engager), TandAbs (tandem diabodies), DAFs (dual acting Fab), two-in-one antibodies, SMIPs (small modular immunopharmaceuticals), FynomAbs (fynomers fused to antibodies), DVD-Igs (dual variable domain immunoglobulin), CovX-bodies (peptide modified antibodies), duobodies and triomAbs. This listing of variants of antibodies and antigen binding fragments thereof is not to be seen as limiting, and a skilled person will be aware of other suitable variants.

In an embodiment of the present invention, the tissue adhesion may be selected from peritoneal adhesion, pericardial adhesion, peritendinous adhesion, and peridural adhesion, but is not limited thereto.

The pharmaceutical composition of the present disclosure may further comprise a pharmaceutically acceptable carrier in addition to the active ingredient integrin α2β1 inhibitor.

The pharmaceutically acceptable carrier contained in the pharmaceutical composition of the present disclosure is ordinarily used at the time of formulation, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil.

The pharmaceutical composition of the present disclosure may further comprise a lubricant, a wetting agent, a sweetening agent, a flavoring agent, an emulsifier, a suspending agent, a preservative, and the like in addition to the above ingredients. Suitable pharmaceutically acceptable carriers and preparations are described in detail in Remington's Pharmaceutical Sciences (19^{th} ed., 1995).

The pharmaceutical composition of the present disclosure may be administered orally or parenterally, for example, by intravenous injection, subcutaneous injection, intramuscular injection, intraperitoneal injection, intrasternal injection, intratumoral injection, topical administration, intranasal administration, intrapulmonary administration, and rectal administration.

The appropriate dose of the pharmaceutical composition of the present disclosure varies depending on factors, such as a formulating method, a manner of administration, patient's age, body weight, gender, and morbidity, food, a time of administration, a route of administration, an excretion rate, and response sensitivity. An ordinarily skilled practitioner can easily determine and prescribe an effective dose (pharmaceutically effective amount) for desired treatment or prevention. According to a preferable embodiment of the present disclosure, the daily dose of the pharmaceutical composition of the present disclosure is 0.0001-100 mg/kg. As used herein, the term “pharmaceutically effective amount” refers to an amount sufficient to prevent or treat the above-described diseases.

As used herein, the term “prevention” refers to a prophylactic or protective treatment of a disease or a disease condition. As used herein, the term “treatment” refers to a reduction, suppression, relief, or eradication of a disease condition.

The pharmaceutical composition of the present disclosure may be formulated into a unit dosage form or may be prepared in a multi-dose container by using a pharmaceutically acceptable carrier and/or excipient according to a method that can be easily implemented by a person having an ordinary skill in the art to which the present disclosure pertains. Here, the formulation may be prepared into a medicine for internal use, an injection, a gel, a film, a perfusate, a spray, an atomizing or vaporizing liquid, a foaming aerosol, an infusion, etc., or may be in the form of a solution in an oily or aqueous medium, a suspension, an emulsion, an extract, a pulvis, a suppository, a powder, a granule, a tablet, or a capsule, and may further contain a dispersant or a stabilizer.

When being in the formulation form of a gel or film, the pharmaceutical composition may be used to cover a target site or may be applied to a target site during or after surgical operation.

When being in the formulation form of a perfusate, the pharmaceutical composition may be perfused into a target site during or after surgical operation.

When being in the formulation form of a spray liquid, the pharmaceutical composition may be provided to or coated at a predetermined thickness on a broad site including a target site during surgical operation.

When being in the formulation form of an atomizing or vaporizing liquid, the pharmaceutical composition may be broadly and uniformly applied as mist to a target site.

When being in the formulation form of a foaming aerosol, the pharmaceutical composition may be broadly and uniformly provided to or coated at a predetermined thickness on a target site.

When being in the formulation form of an infusion, the pharmaceutical composition may be administered by instillation before, during, or after surgical operation.

In addition, the pharmaceutical composition of the present disclosure may comprise another pharmaceutically active agent or drug, for example, a therapeutic agent such as mitomycin-C, rosuvastatin, etc.

According to another aspect thereof, the present disclosure provides a method for screening a material for prevention or treatment of tissue adhesion, the method comprising the following steps of:

(a) co-culturing macrophage-like THP-1 cells with collagen I-expressing cells or cells transformed to express collagen I;

(b) treating the co-cultured collagen I-expressing cells with a candidate material;

(c) measuring an expression level of an integrin α2β1 protein or gene in the collagen I-expressing cell treated with the candidate material; and

(d) determining the candidate material to be a material for prevention or treatment of tissue adhesion when the measured expression level of the integrin α2β1 protein or gene in step (c) is reduced, compared to untreated, collagen I-expressing cells.

The screening method for a material for prevention or treatment of tissue adhesion according to the present disclosure will be explained in a stepwise manner.

Step (a): Co-Culturing Macrophage-Like THP-1 Cells with Collagen I-Expressing Cells or Cells Transformed to Express Collagen I

As used herein, the term “culturing” refers to growing partial organs, tissues, or cells of an organism in an artificial condition.

As used herein, the term “co-culturing” means culturing two or more kinds of cells in combination.

In the present disclosure, co-culturing macrophage-like THP-1 cells with collagen I-expressing cells is intended to mimic a condition inducing an inflammatory response in a human tissue.

The term “collagen I-expressing cells”, as used herein, refers to cells that express or secrete collagen I as an extracellular matrix component. Examples of the collagen I-expressing cells include dura mater cells and fibroblasts, but are not limited thereto.

The term “dura mater cells”, as used herein, refers to cells constituting the outermost of the three layers of the thick and tough membrane made of connective tissue that surrounds the brain and spinal cord. More particularly, the dura mater cells may be derived from mammals, for example, Monotremata, Marsupialia, Edentate, Dermoptera, Chiroptera, Camivora, Insectivora, Proboscidea, Perissodactyla, Artiodactyla, Tubulidentata, Pholidota, Sirenia, Cetacean, Primates, Rodentia, and Lagomorpha. Preferably, the dura mater cells may be derived from humans.

THP-1 cells, which are human monocyte cells deposited as ATCC TIB-202, are differentiated into macrophage-like cells that exhibit phagocytic activity and secretion of proinflammatory cytokines, in the presence of phorbol 12-myristate 13-acetate (PMA). Here, THP-1 cells are called macrophage-like cells, macrophage-like THP-1 cells, macrophages, or MΦ.

Step (b): Treating the Co-Cultured Collagen I-Expressing Cells with Candidate Material

As used herein, the term “candidate material” refers to a test agent and a material interchangeably used with the test agent and is intended to encompass any substance, molecule, element, compound, entity, or a combination thereof. For example, proteins, polypeptides, small organic molecules, polysaccharides, polynucleotides, and so on may be used. In addition, a candidate material may be a natural product or a combination of synthetic compounds.

As used herein, the term “treating” means a physical contact collagen I-expressing cells with a candidate material. The contacting can be achieved by adding a candidate material to a culture medium for culturing collagen I-expressing cells and incubating the cells for a predetermined time.

Step (c): Measuring Expression Level of Integrin α2β1 Protein or Gene in Collagen I-Expressing Cells Treated with Candidate Material or Binding Affinity of Integrin α2β1 for Collagen I

In this disclosure, the collagen I-expressing cells co-cultured with macrophage-like cells mimic a condition of tissues inducing an inflammatory response. As proven in an embodiment of the present disclosure, collagen I-expressing cells in which an inflammatory response has been induced increases in the expression level of integrin α2β1 and in the binding affinity of integrin α2β1 for collagen I.

An increase in the expression level of integrin α2β1 or in the binding affinity of integrin α2β1 for collagen I contributes to the adhesion of tissues. Hence, an increase or decrease in the expression level of integrin α2β1 or in the binding affinity of integrin α2β1 for collagen I indicates an effect of the candidate material on the adhesion of tissues.

Unless specially stated otherwise, the term “measuring an expression level of a protein or gene” means qualitative or qualitative detection of a gene or protein in a corresponding sample.

In the present disclosure, measuring an expression level of a gene may be achieved by detecting the transcript mRNA or the translation product protein of a gene in a sample.

The detection of mRNA or protein can be typically implemented by an RNA or protein from a sample and subjecting the same to reverse transcription polymerase chain reaction (RT-PCR), competitive RT-PCR, real-time RT-PCR, RNase protection assay (RPA), northern blotting, DNA chip, western blotting, enzyme-linked immunosorbent assay (ELISA), radioimmunoassay (RIA:), radioimmunodiffusion, Ouchterlony immunodiffusion, rocket immunoelectrophoresis, tissue immunostaining, immunoprecipitation assay, complement fixation assay, fluorescence activated cell sorter (FACS), or protein chip, but with no limitations thereto. Any technique known in the art could be employed.

In the present disclosure, binding affinity of integrin α2β1 for collagen I can be measured by enzyme linked immunosorbent assay (ELISA), immunostaining, adhesion assay, or the like, but with no limitations thereto.

Step (d): Determining the Candidate Material to be a Material for Prevention or Treatment of Tissue Adhesion When the Measured Expression Level of the Integrin α2β1 Protein or Gene in Step (c) is Reduced, Compared to Untreated, Collagen I-Expressing Cells

Here, the term “reduction” used in conjunction with expression levels of proteins or genes accounts for the case where i) an expression of integrin α2β1 protein or gene is lower than that in a sample acquired from cells not treated with a candidate material or ii) a binding affinity of integrin α2β1 for collagen I is lower than that in a sample acquired from cells not treated with a candidate material.

In an embodiment of the present disclosure, the tissue adhesion may be selected from peritoneal adhesion, pericardial adhesion, peritendinous adhesion, and peridural adhesion, but is not limited thereto.

According to a further aspect thereof, the present disclosure provides an anti-adhesion composition comprising the pharmaceutical composition and a biocompatible hydrogel.

In addition, the anti-adhesion composition of the present disclosure may be used in the form of a gel, a film, or a membrane, but with no limitations thereto.

In an embodiment of the present disclosure, the integrin α2β1 inhibitor is selected from the group consisting of an antibody, an aptamer, a natural extract, and a compound. In an embodiment of the present disclosure, the integrin α2β1 inhibitor is selected from an anti-integrin α2β1 antibody or an antigen-binding fragment thereof, BTT 3016, BTT 3033, BTT 3034, and rhodocetin.

In an embodiment of the present disclosure, the adhesion may be selected from peritoneal adhesion, pelvic adhesion, pericardial adhesion, peritendinous adhesion, and peridural adhesion, but is not limited thereto.

In an embodiment of the present disclosure, the anti-adhesion composition may be applied to any portion of the human body, especially to various intraperitoneal and intrathoracic viscera, epitendineum, cranium, nerve, and eye balls upon laparotomy, gynecologic operation, and thoracotomy, to tendons and ligaments upon orthopedic operation, and to dura mater upon neurosurgery.

In an embodiment of the present disclosure, the hydrogel may include a biocompatible polymer selected from the group consisting of hyaluronic acid, collagen, gelatin, carboxymethyl cellulose, propylene glycol, polyethylene glycol, hydroxypropyl cellulose (HPC), hydroxypropylmethyl cellulose (HPMC,), polycaprolactone (PCL), polyglycolide (PGA), polylactic acid (PLA), polylactide-glycolide copolymer (PLGA), polyvinylpyrrolidone (PVP), polyethylene oxide (PEO,), polypropylene oxide (PPO), polyvinylmethyl ether (PVME), polymethyl acrylate (PMA), polyesteramide, polybutyric acid, acrylamide (acrylic amide), acrylic acid, alginic acid, pectin, carrageenan, chondroitin sulfate, dextran sulfate, chitosan, polylysine, carboxymethyl chitin, fibrin, agarose, pullulan, polyanhydride, polyorthoester, polyetherester, polyesteramide, polyvaleric acid, polyacrylate, ethylene-vinyl acetate polymer, acryl-substituted cellulose acetate, polyvinyl chloride, polyvinyl fluoride, polyvinyl imidazole, chlorosulphonate polyolefins, polyvinylpyrrolidone (PVP), hydroxypropylmethyl cellulose (HPMC), ethyl cellulose (EC), hydroxypropyl cellulose (HPC), and a combination thereof, but without limitations thereto.

According to a further aspect thereof, the present disclosure provides a method for preventing or treating tissue adhesion, the method comprising a step of administering the pharmaceutical composition comprising an integrin α2β1 inhibitor as an active ingredient to a subject.

As used herein, the term “administrating” or “administer” refers to the direct application of a therapeutically effective amount of the composition of the present disclosure to a subject (individual) in need of the composition, thereby forming the same amount thereof in the body of the subject.

The term “therapeutically effective amount” of the composition refers to the content of the composition, which is sufficient to provide a therapeutic or prophylactic effect to a subject, to which the composition is to be administered, and thus the term has a meaning encompassing “prophylactically effective amount”.

As used herein, the term “subject” includes, but is not limited to, a human being, mouse, rat, guinea pig, dog, cat, horse, cow, pig, monkey, chimpanzee, baboon, or rhesus monkey. Specifically, the subject of the present invention is a human being.

In an embodiment of the present disclosure, the integrin α2β1 inhibitor is selected from the group consisting of an antibody, an aptamer, a natural extract, and a compound.

In an embodiment of the present disclosure, the integrin α2β1 inhibitor is selected from an anti-integrin α2β1 antibody or an antigen-binding fragment thereof, BTT 3016, BTT 3033, BTT 3034, and rhodocetin.

In an embodiment of the present disclosure, the adhesion may be selected from peritoneal adhesion, pelvic adhesion, pericardial adhesion, peritendinous adhesion, and peridural adhesion, but is not limited thereto.

In an embodiment of the present disclosure, the pharmaceutical composition comprises a hydrogel.

In an embodiment of the present disclosure, the pharmaceutical composition may be in the form of a formulation selected from the group consisting of a medicine for internal use, an injection, a gel, a film, a perfusate, a spray liquid, an atomizing or vaporizing liquid, a foaming aerosol, and an infusion.

According to still another aspect thereof, the present disclosure provides a method for prevention or treatment of adhesion tissue, the method comprising a step of administering an anti-adhesion composition comprising an integrin α2β1 inhibitor and a biocompatible hydrogel.

In an embodiment of the present disclosure, the anti-adhesion composition is in the form of a gel or film.

Because the method for prevention or treatment of tissue adhesion according to the present disclosure comprises a step of administering the pharmaceutical composition or the anti-adhesion composition according to an aspect of the present disclosure, the common descriptions between them are omitted in order to avoid undue redundancy leading to the complexity of this specification.

### Advantageous Effects

Features and advantages of the present disclosure are summarized as follows:

(a) the present disclosure provides a pharmaceutical composition comprising an integrin α2β1 inhibitor as an active ingredient for prevention or treatment of tissue adhesion;

(b) a material preventive of or therapeutic for tissue adhesion can be screened using the screening method of the present disclosure;

(c) the present disclosure provides an anti-adhesion composition comprising the pharmaceutical composition for prevention or treatment of tissue adhesion and a biocompatible hydrogel; and

(d) the present disclosure provides a method for prevention or treatment of tissue adhesion, the method comprising a step of administering the pharmaceutical composition for prevention or treatment of tissue adhesion to a subject.

### BRIEF DESCRIPTION OF THE DRAWINGS

FIG. 1 shows the sufficient attachment of human dura mater tissues to a collagen-coated culture dish under the load of cell culture plates for 3 days.

FIG. 2 shows time-lapse microscopic images of hDMCs on days 3, 7, and 14;

FIG. 3 shows microscopic images of hDMCs after immunocytochemical staining for vimentin, cytokeratin, GFAP, and synaptophysin;

FIG. 4 shows microscopic images hDMCs after immunocytochemical staining for S-100 and neuron-specific enolase;

FIG. 5 shows cell viability and flow cytometry analysis results between naïve and co-cultured hDMCs;

FIG. 6 is an overall view of adhesion assay results;

FIG. 7 shows quantitative analysis results of adhesion of naïve hDMCs to extracellular matrix components;

FIG. 8 shows quantitative analysis results of naïve hDMCs and co-cultured hDMCs to extracellular matrix components;

FIG. 9 shows expression levels of focal adhesion kinase (FAK) in naïve hDMCs and co-cultured hDMCs as measured by western blotting;

FIG. 10 shows quantitative analysis results of expression levels of focal adhesion kinase (FAK) in naïve hDMCs and co-cultured hDMCs;

FIG. 11 shows cell viability of naïve hDMCs as measured by flow cytometry;

FIG. 12 shows expression patterns of integrin subtypes in naïve hDMCs as measured by immunofluorescence flow cytometry;

FIG. 13 shows expression patterns of integrin subtypes in naïve hDMCs and co-cultured hDMCs as measured by immunofluorescence flow cytometry;

FIG. 14 shows quantitative analysis results of expression patterns of integrin subtypes in naïve hDMCs and co-cultured hDMCs as measured by immunofluorescence flow cytometry;

FIG. 15 shows changes in cell viability of hDMCs by treatment with BTT 3033;

FIG. 16 shows quantitative assay for adhesion ability of co-cultured hDMCs treated with BTT 3033;

FIG. 17 shows levels of MMP, TIMP, and VEGF in culture media of naïve hDMCs and co-cultured hDMCs as measured by ELISA;

FIG. 18 shows relative levels of MMP, TIMP, and VEGF on the basis of log₂ (co-cultured cells/[hDMCs+MΦ]);

FIG. 19 shows quantitative real-time PCR analysis of MMP-1 and MMP-3 in naïve and co-cultured cells of MΦ and hDMCs, as measured for relative expression level ratios; and

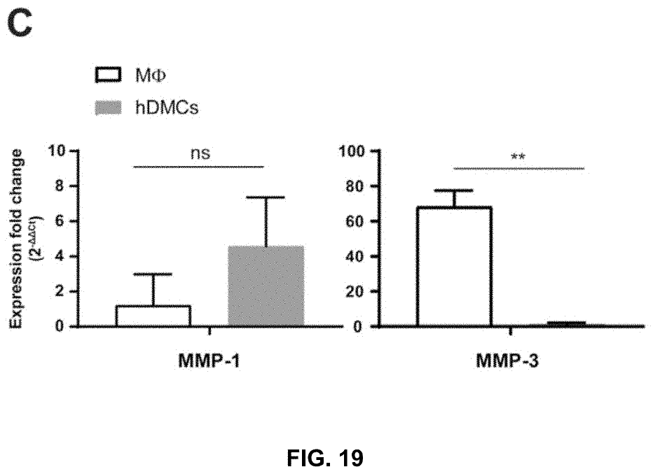

FIG. 20 is a schematic illustration of hDMC adhesion to ECM during inflammation.

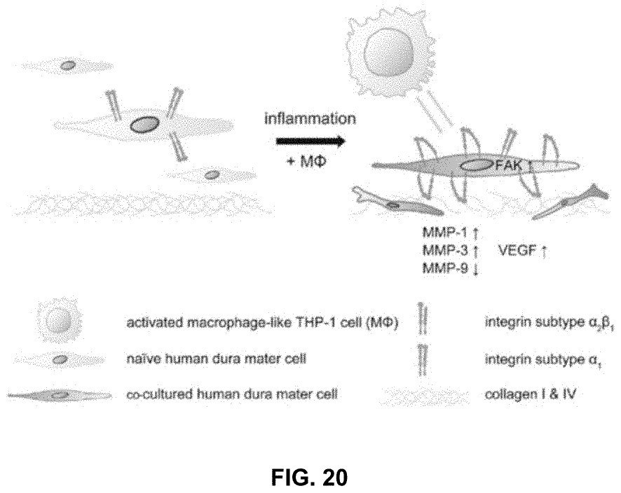

### DETAILED DESCRIPTION

A better understanding of the present disclosure may be obtained through the following examples which are set forth to illustrate, but are not to be construed as limiting the present disclosure.

### EXAMPLES

### Example 1

### Collection of Human Dura Mater Tissue

Human dura mater tissues were obtained from patients who had undergone neurosurgical procedures. Specimens were collected from 10 patients with the following characteristics (Table 1, eight males and two females, mean age 53.0±21.0 years; four pieces of 1 cm² dura mater from each patient).

TABLE 1

| | | | | | Past medical history | | | | |
|---|---|---|---|---|---|---|---|---|---|
| | | | | Surgical | Diabetes | Hyper | | | |
| No. | Age | Sex | Diagnosis | operation | Mellitus | tension | Alcohol | Smoking | Others |
| 1 | 20 | F | acute subdural | decompressive | − | − | − | − | − |
| | | | hemorrhage with | craniectomy and | | | | | |
| | | | skull fracture | duroplasty | | | | | |
| 2 | 61 | M | cerebral infarction | decompressive | − | − | + | + | arrhythmia |
| | | | with intracranial | craniectomy and | | | | | |
| | | | hemorrhage | duroplasty | | | | | |
| 3 | 78 | F | subarachnoid | decompressive | − | + | − | − | − |
| | | | hemorrhage | craniectomy and | | | | | |
| | | | | duroplasty | | | | | |
| 4 | 58 | M | cerebral infarction | decompressive | − | + | + | + | Hypo-thyroidism |
| | | | | craniectomy and | | | | | |
| | | | | duroplasty | | | | | |
| 5 | 19 | M | epidural | decompressive | − | − | − | − | − |
| | | | hemorrhage with | craniectomy and | | | | | |
| | | | hemorrhagic | duroplasty | | | | | |
| | | | contusion | | | | | | |
| 6 | 71 | M | intracranial | decompressive | − | − | + | + | benign |
| | | | hemorrhage with | craniectomy and | | | | | prostatic |
| | | | intraventricular | duroplasty | | | | | hyper-plasia |
| | | | hemorrhage | | | | | | |
| 7 | 53 | M | acute subdural | decompressive | − | + | + | + | − |
| | | | hemorrhage with | craniectomy and | | | | | |
| | | | hemorrhagic | duroplasty | | | | | |
| | | | contusion | | | | | | |
| 8 | 51 | M | acute subdural | decompressive | − | − | + | + | − |
| | | | hemorrhage with | craniectomy and | | | | | |
| | | | skull fracture | duroplasty | | | | | |
| 9 | 77 | M | acute subdural | decompressive | + | − | + | − | − |
| | | | hemorrhage | craniectomy and | | | | | |
| | | | | duroplasty | | | | | |
| 10 | 42 | M | epidural | decompressive | − | − | − | − | − |
| | | | hemorrhage with | craniectomy and | | | | | |
| | | | skull fracture | duroplasty | | | | | |

All specimens were immediately placed in DMEM (Welgene, Gyeongsangbuk-do, Republic of Korea) at 4° C. and transferred to the laboratory.

This study adhered to the guidelines and protocols approved by the local institutional review board of the hospital of the present inventors.

### Example 2

### Immunostaining

The cells were fixed in 4% (w/v) paraformaldehyde (Electron Microscopy Sciences; Hatfield, Pa.) for 10 min at 37° C. and washed with PBS (pH 7.4; Welgene) before the cell membrane was permeabilized by addition of 0.5% (v/v) Triton X-100 (Sigma-Aldrich) in PBS for 10 min at room temperature. After being washed with PBS, the cells were immersed in PBS containing 1% (w/v) bovine serum albumin (Sigma-Aldrich), 22.52 mg/ml glycine (Sigma-Aldrich), and 0.1% (v/v) Tween 20 (Sigma-Aldrich) for 30 min to block non-specific binding.

The primary antibodies used for immunostaining were antibodies against vimentin (M7020, 1:100; Dako, Agilent, Santa Clara, Calif., USA), cytokeratin and GFAP (glial fibrillary acidic protein) (M3515, M0761, 1:100; Dako), synaptophysin (CMC336A, 1:100; Cell Marque, Darmstadt, Germany), S-100 (sc-53438, 1:100; Santa Cruz Biotechnology, Dallas, Tex., USA), and enolase (sc-51880, 1:100; Santa Cruz Biotechnology). Alexa Fluor 488 goat anti-mouse and anti-rabbit IgG antibodies were used as secondary antibodies.

For nuclear staining, 4′, 6-diamidino-2-phenylindole (DAPI)(Thermo Fisher Scientific, Waltham, Mass., USA) was used.

Optical microscopic images were obtained using an Olympus IX71 inverted microscope and DP70 camera (with UPlanFL 4×/0.13 numerical aperture and LUCPlanFL 10×/0.30 numerical aperture objective lens) (Olympus; Tokyo, Japan).

Fluorescence and confocal laser scanning images were acquired using a Carl Zeiss Axio Observer Z1, AxioCam HRc camera, and LSM700 system (with EC Plan-Neofluar 10×/0.30 numerical aperture objective lens) (Carl Zeiss, Göttingen, Germany). For imaging, 405 nm and 488 nm continuous-wave lasers and an HBO 100 illuminator were coupled with 420-465 nm and 500-550 nm filters (Carl Zeiss). ImageJ software was used for image processing.

### Example 3

### Human Dura Mater Cell (hDMC) Culture

Primary Culture Method

Dura mater tissues were washed three times with Hank's balanced saline solution (HBSS) [containing 1% (w/v) penicillin/streptomycin (P/S; Gibco, Grand Island, N.Y., USA)] to remove any blood clots and other contaminants. Then, the tissues were placed on a collagen-coated 100 mm culture dish, with the outer portion of the dura mater facing downward. The tissues were incubated in DMEM [containing 10% (v/v) fetal bovine serum (Gibco), 1% (w/v) P/S, and 1% (v/v) MEM nonessential amino acid supplement (Gibco)] at 37° C. in a 5% (v/v) carbon dioxide atmosphere. The cells were detached with Accutase (BD Biosciences, San Jose, Calif., USA) solution, followed by multiple washes with HBSS to isolate confluent cells. If the cells were not needed immediately, they were preserved in liquid nitrogen and then thawed using conventional methods. The isolated or thawed hDMCs were plated at a density of 2.0×10⁴ cells/well into collagen I-coated 6-well plates, with each well containing 4 mL medium. Cultivation of the tissues or cells was performed at 37° C. in a humidified atmosphere containing 5% (v/v) carbon dioxide. Collagen-coated dishes or plates were used for all procedures to cultivate dura mater tissues and hDMCs. Collagen-coated dishes and plates were prepared by adding 5 μg/cm² bovine collagen I (Gibco) at a concentration of 50 μg/mL to 20 mM acetic acid. After the dishes or plates were left at room temperature for 1 hour, the solution was carefully aspirated, and the dishes or plates were washed three times with HBSS. The dishes and plates were either used immediately or stored at 4° C. until use.

Culture Results

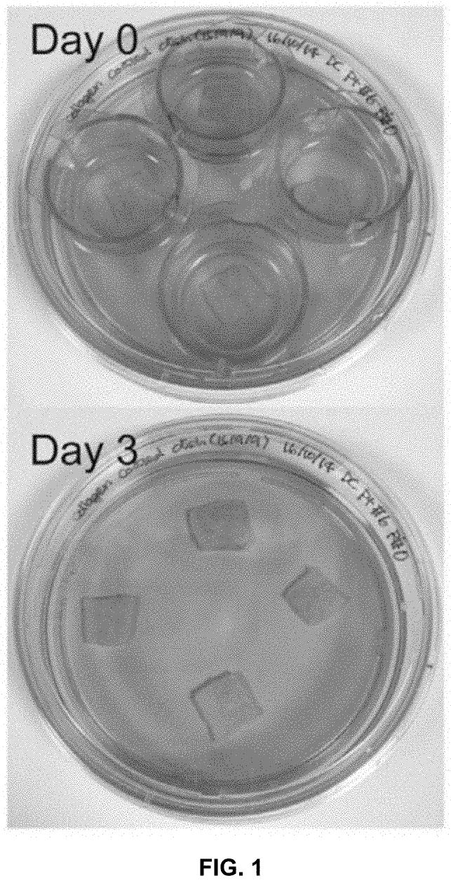

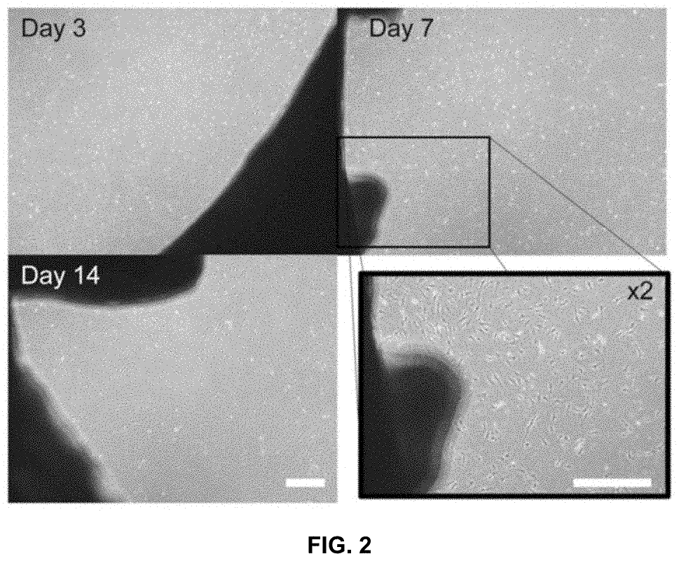

The human dura mater tissue adhered sufficiently to the collagen-coated dishes and plates with 3 days of gentle compression (FIG. 1). After 3 days, hDMCs, which had a similar morphology to fibroblasts, appeared at the edge of the explanted tissues. The cells initially became elongated or formed multiple protruding pseudopods before migrating away from the tissue. After an average of 14 days, full confluence of the hDMCs was achieved with good viability (FIG. 2). Even after the frozen cells were thawed, the hDMCs had good viability with no specific alterations in morphology or growth until the fourth or fifth passage.

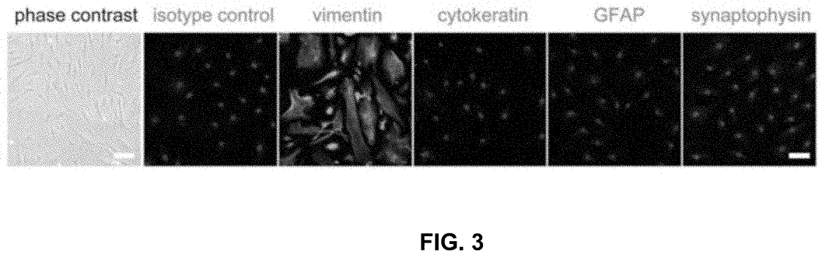

The isolated cells were strongly positive for vimentin, negative for cytokeratin and GFAP (glial fibrillary acidic protein), and weakly positive for synaptophysin, which implies that these cells had a fibroblast-like phenotype (FIG. 3).

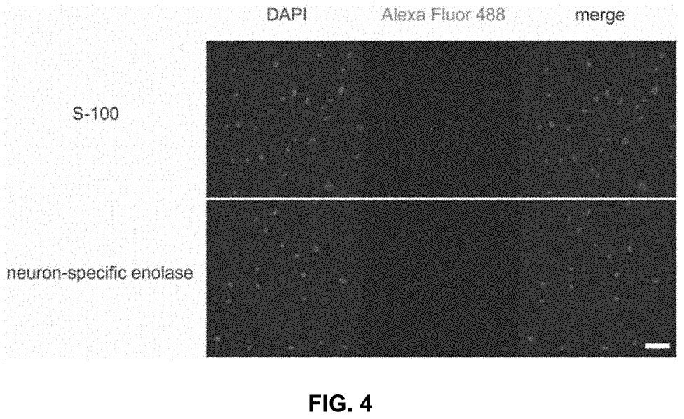

In addition, the isolated cells were negative for S-100 protein and neuron-specific enolase. Based on the results, it was concluded that neuronal phenotype was not presented in the isolated cells (FIG. 4).

### Example 4

### Co-Culture of Activated Macrophage-Like THP-1 Cells (MΦ) and Human Dura Mater Cells (hDMCs)

The human acute monocytic leukemia cell line THP-1 (Korea Cell Line Bank, Republic of Korea) was maintained in RPMI-1640 medium (Welgene) supplemented with 10% (v/v) fetal bovine serum (FBS, Gibco, Grand Island, N.Y.), 1% (w/v) penicillin/streptomycin (P/S; Gibco), and 0.05 mmol/L 2-mercaptoethanol (Sigma-Aldrich).

In order to differentiate THP-1 cells into macrophage-like cells, the medium was replaced with 4 ml DMEM [containing 10% (v/v) FBS, 1% (w/v) P/S, and 160 nmol/L phorbol 12-myristate 13-acetatate] 72 hours prior to co-culture and analysis.

For all experiments, hDMCs from the first or second passage were detached using Accutase (BD Biosciences, San Jose, Calif.), washed twice with HBSS, and plated into six-well plates at 2.0×10⁴ cells/well in 4 ml DMEM [containing 10% (v/v) FBS, 1% (w/v) P/S]. After 3 days, the medium was changed to 4 mL DMEM [containing 1% (v/v) FBS, 1% (w/v) P/S], and activated macrophage-like THP-1 cells (MΦ) were detached using Accutase and added to the six-well culture plates in 1 ml DMEM containing 1% (v/v) FBS, 1% (w/v) P/S. After 24 hours, the medium was collected and stored at −80° C., and the cells were used for further experiments.

4-1. Phenotypic Identification of THP-1 and Induction of Inflammation Response in Co-Cultured hDMCs

For phenotypic analysis of THP-1, direct immunofluorescence flow cytometric assay was conducted. FACS was performed using the LSR Fortessa X-20 cell analyzer (BD Biosciences, San Jose, Calif., USA) (FlowJo ver. 10 software (FlowJo, Ashland, Oreg., USA)).

For direct immunofluorescence flow cytometric assays, anti-CD14 mouse monoclonal IgG₁-fluorescein isothiocyanate (ab28061; Abcam), anti-CD80 mouse monoclonal IgG₁-phycocyanin (ab69778; Abcam), anti-CD163 mouse monoclonal IgG₁-PE/Cy7 (ab233653; Abcam), and anti-mannose receptor (CD206) rabbit monoclonal IgG-allophycocyanin (ab223961; Abcam) antibodies were used.

As a result, macrophage-like THP-1 cells (MΦ) showed decreased expression levels of CD14, CD163, and CD206 and an increased expression level of CD80 (Table 2). Macrophages are divided into M1 and M2 macrophages. M1 macrophages encourage inflammation and can be identified by their specific expression of the markers CD40, CD80, and CD86 while M2 macrophages show an anti-inflammatory effect and are characterized by the expression of the markers CD163 and CD206.

That is, the results indicate that the MΦ of the present invention has a characteristic of M1 macrophages rather than the M2 macrophages.

TABLE 2

| | THP-1 cell | Mϕ | p value |
|---|---|---|---|
| CD14 | 1.000 ± 0.075 | 0.912 ± 0.078 | p < 0.001 |
| CD80 | 1.000 ± 0.920 | 1.176 ± 0.160 | p < 0.001 |
| CD163 | 1.000 ± 0.047 | 0.498 ± 0.038 | p < 0.001 |
| CD206 | 1.000 ± 0.034 | 0.262 ± 0.028 | p < 0.001 |

In order to determine whether an inflammatory response was induced in the co-cultured human dura mater cells, ELISA was performed for inflammatory cytokine change. Inflammatory responses were assayed for IL-1β, IL-6, IL-12, and TNF-α using DuoSet ELISA Development System kits (IL-1b: DY201, IL-6: DY206, IL-12: DY1270, TNF-α: DY210).

As understood from the ELISA data, TNF-α showed about 4-fold increased expression in the co-culture compared to the simple sum of the human dura mater cells and MΦ (Table 3).

TABLE 3

| | | | hDMCs + | | Fold |
|---|---|---|---|---|---|
| | hDMCs | Mϕ only | Mϕ only | Co-culture | change |
| IL-1β (pg/mL) | 2.19 ± 1.19 | 3.40 ± 0.51 | 5.59 ± 0.51 | 3.87 ± 1.24 | 0.69 ± 0.22 |
| IL-6 (pg/mL) | 6.87 ± 3.11 | 3.67 ± 1.48 | 10.5 ± 1.48 | 16.4 ± 7.54 | 1.56 ± 0.72 |
| TNF-α (pg/mL) | 0.94 ± 0.29 | 0.90 ± 0.53 | 1.83 ± 0.53 | 7.25 ± 0.63 ** | 3.96 ± 0.34 |

** p < 0.01 (n = 3-6 per group, unpaired two-tailed t-test). All data are means ± standard error of the mean.

Taken together, the data of phenotype analysis for THP-1 cells and ELISA for secretary cytokines suggest that the co-culture of THP-1 cells and hDMCs had successfully induced an inflammatory response.

4-2. Cytotoxicity Assay of Co-Culture of Activated Macrophage-Like THP-1 (MΦ) and hDMCs

To assay adverse effects of inflammatory responses induced by the co-culture, measurements were made of cell viability of naïve hDMCs, MΦ, and co-cultured hDMCs.

To evaluate cell viability, EZ-Cytox cell viability assay kit (Daeillab Service Ltd., Seoul, Republic of Korea) was used according to the manufacturer's instructions. Water-soluble tetrazolium salt (WST) reagent was added to each well. After 1 hour of incubation, the absorbance of the supernatant was measured at the wavelength of 450 nm on a microplate absorbance reader. The viability was assessed by normalized absorbance values.

Furthermore, flow cytometry was used as an additional experiment to evaluate cell viability. Reference may be made to Example 6 with respect to flow cytometry.

As measured by the cell viability assay kit, cell viability of co-cultured hDMCs was reduced by 28.0% compared to the naïve (1.00±0.09 for naïve, 0.72±0.12 for co-cultured; p<0.001).

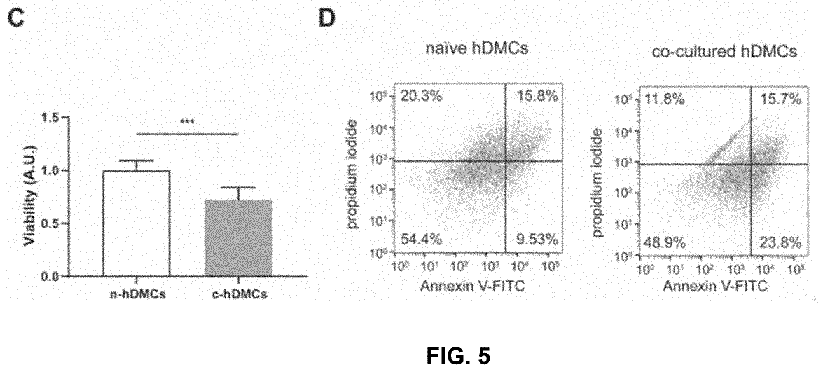

However, flow cytometry analysis showed a 5.5% decrease in live cells ratio of the co-cultured hDMCs, compared the naïve cells (54.4% for naïve and 48.9% for co-cultured) (FIG. 5).

The serum starvation process which had performed 24 hours before the initiation of co-culturing and the ratio increment of early apoptosis cells upon co-culture (9.53% for naïve and 23.8% for co-culture) might have an influence on the result of cell viability assay.

Based on results of the flow cytometry, the inflammatory response induced by the co-culture might not greatly influence the apoptosis and might be acceptable to proceed the following experiments.

### Example 5

### Assay for Adhesion of hDMCs to ECM

Collagen is the most abundant protein in vivo, and it plays major roles in cellular function. For example, collagen I is a major extracellular matrix (ECM) component produced by fibroblasts during wound healing, and collagen IV is involved in various cellular interactions. In addition, fibronectin, which is produced by both fibroblasts and macrophages, also contribute to fibroblast chemotaxis during inflammation.

Hence, evaluation was made of the involvement of fibronectin, collagens I and IV, laminin I, and fibrinogen, which are major components of the ECM related to cell adhesion, in hDMCs.

5-1. Adhesion Assay for Ability of hDMCs to Adhere to ECM

Adhesion assays identifying ECM components adhering to hDMC were performed using CytoSelect 48-well cell adhesion assay kits (Cell Biolabs Inc., San Diego, Calif.).

After detachment of naïve hDMCs and MΦ-co-cultured hDMCs, cell suspensions were prepared in serum-free DMEM. A total of 1.5×10³ cells in 150 μl serum-free medium were added to each well and incubated for 1 hour at 37° C. After removal of the medium, the cells were washed four times with 150 μl PBS.

Cell staining and protein extraction were performed according to the manufacturer's instructions. Cellular adhesion was assessed by measuring the optical density at 560 nm on a Gemini XPS Microplate Reader (Molecular Devices, Sunnyvale, Calif.).

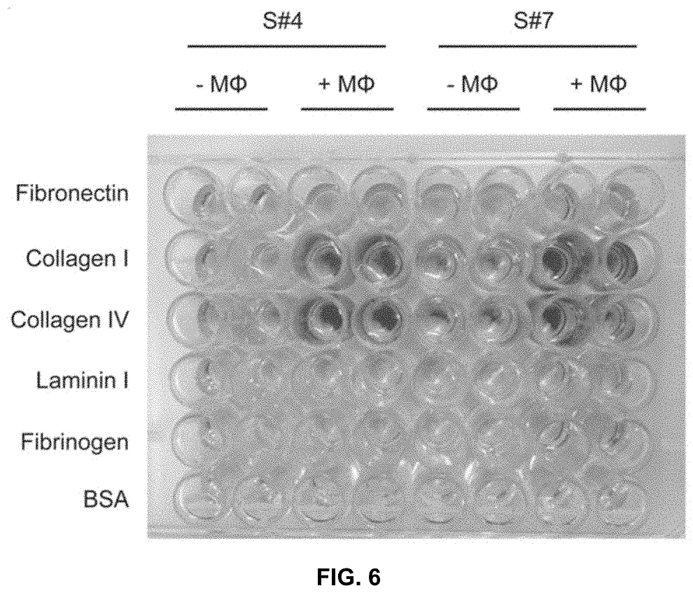

The naïve hDMCs exhibited partial staining for collagen I and IV in the adhesion assay. In addition, increased staining for collagen I and IV was observed in the hDMCs co-cultured with MΦ, compared to the naïve hDMCs (FIG. 6).

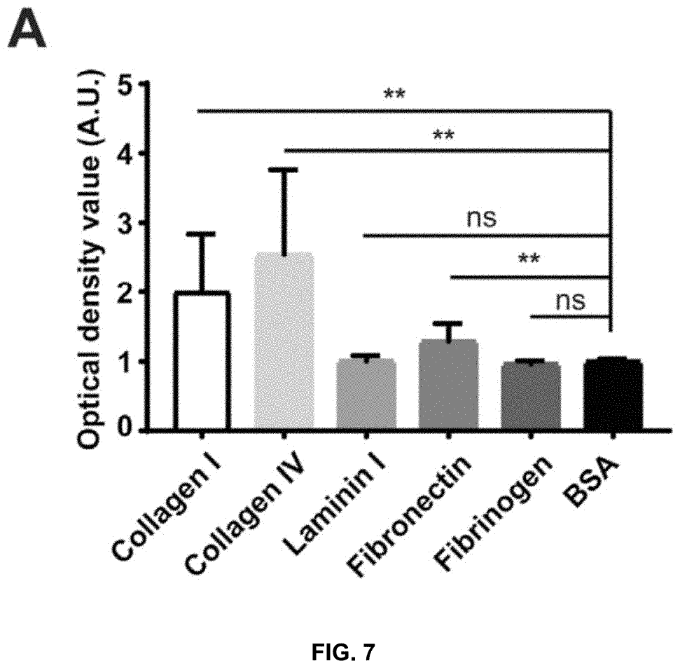

Absorbance analysis quantitated the adhesion of naïve hDMCs to EMC components, revealing that the naïve hDMCs adhered through collagen I (2.00±0.84), collagen IV (2.55±1.22), and fibronectin (1.29±0.25) (p<0.01), but with no significant values for laminin I (1.01±0.08) or fibrinogen (0.96±0.05) (FIG. 7).

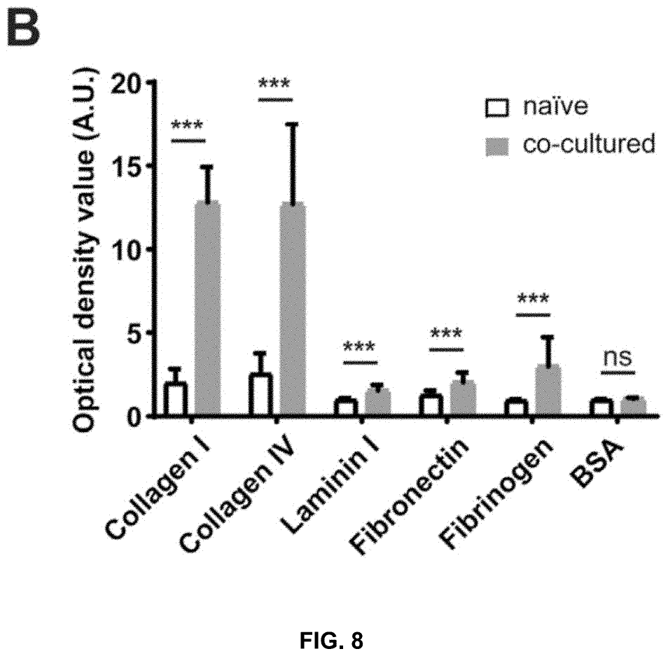

Adhesion of hDMCs was increased through all ECM components in the MΦ-co-cultured hDMCs (FIG. 8, p<0.001). Specifically, the adhesion of the co-cultured hDMCs increased by 6.4-fold (12.80±2.12) through collagen I and by 5.0-fold (12.704±79) through collagen IV, 1.6-fold (2.04±0.56) through fibronectin, by 1.6-fold (1.55±0.39) through laminin I, and by 3.1-fold (2.99±1.74) through fibrinogen, compared with the adhesion of naïve cells.

5-2. Western Blot Analysis for FAK Expression in Co-Cultured hDMCs

To specifically determine whether inflammation increases the adhesion of hDMCs, levels of focal adhesion kinase (FAK), which is a factor associated with cell adhesion, migration, and viability were analyzed via western blotting.

Cell lysates from naïve hDMCs and MΦ-co-cultured hDMCs were subjected to western blot analysis to evaluate the expression of FAK.

As primary antibodies for the western blotting, an anti-FAK antibody (ab40794, 1:1,000; Abcam, Cambridge, Mass., USA), and anti-GAPDH antibody (sc-47724, 1:1,000; Santa Cruz Biotechnology, Dallas, Tex., USA) were used.

The secondary antibodies were horseradish peroxidase (HRP)-conjugated goat anti-rabbit and anti-mouse polyclonal IgG antibodies (anti-rabbit: GTX213110-01, 1:500; anti-mouse: GTX213111-01, 1:500; GeneTex, Irvine, Calif., USA).

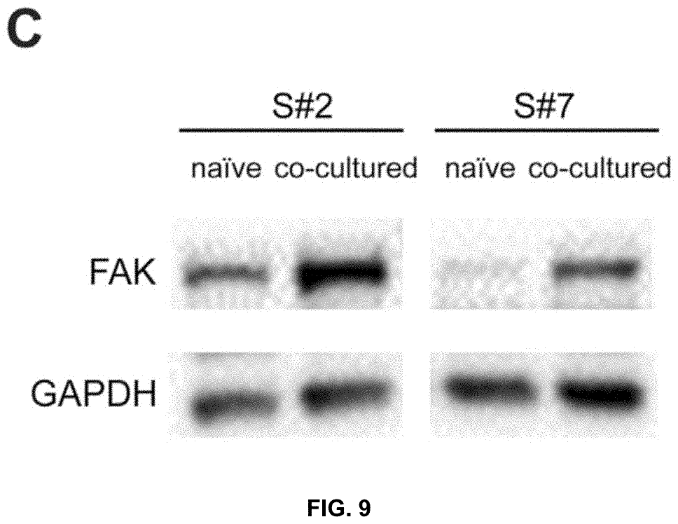

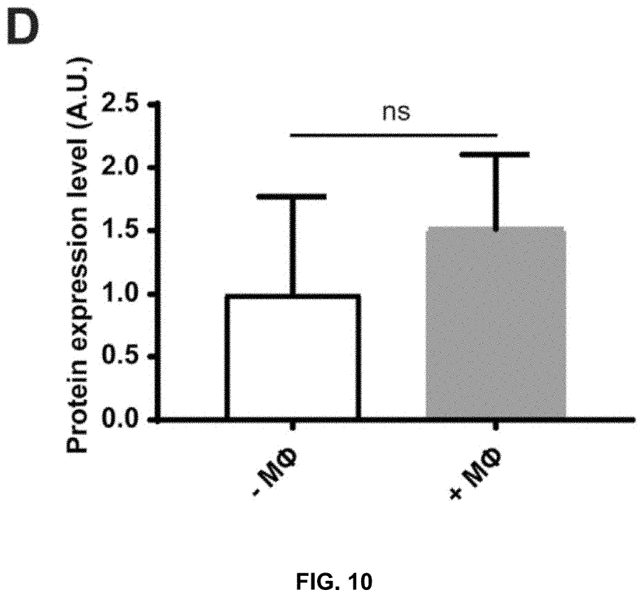

FAK expression increased about 1.5-fold in the co-cultured cells (1.51±0.59) compared to the naïve cells (1.00±0.77) (FIGS. 9 and 10). The data indicate that an increase in FAK level of the co-cultured cells drives the activation of cell adhesion.

### Example 6

### Flow Cytometry Assay for Role of Integrin Subtype in hDMC Adhesion Upon Inflammatory Response

Because integrins play an important role in cell adhesion to ECM components, hDMC integrin expression was investigated.

The roles of integrin subtypes in the adhesion of hDMCs in inflammation were analyzed by Annexin V-fluorescein isothiocyanate/propidium iodide apoptosis assay, specifically, by fluorescence-activated cell sorting (FACS) using BD Pharmingen™ FITC Annexin V Apoptosis Detection Kit I (CAT No. 556547, BD Biosciences). For phenotypic analysis of hDMCs, direct and indirect immunofluorescence labeling and FACS analysis were performed using the LSR Fortessa X-20 cell analyzer (BD Biosciences) running FlowJo ver. 10 software (FlowJo, Ashland, Oreg., USA).

In this regard, the primary antibodies used were anti-integrin α₁ and anti-integrin α₂β₁ (anti-integrin α₁: ab34445, anti-integrin α₂β₁: ab30483; Abcam), anti-integrin α_{V}β₃ (bs-1310R; Bioss Antibodies, Woburn, Mass., USA), and anti-integrin α_{IIb}β₃ (MA1-21188; Thermo Fisher Scientific).

Mouse IgG₁ monoclonal and rabbit IgG monoclonal antibodies (mouse IgG₁: ab81032, rabbit IgG: ab199376; Abcam) were used for the isotype control.

The secondary antibodies included goat anti-mouse IgG APC and anti-rabbit IgG-APC polyclonal IgG antibodies (anti-mouse IgG-APC: A865, anti-rabbit IgG-APC: A-10931; Thermo Fisher Scientific).

6-1. Assay for Cell Viability of Naïve hDMCs by Flow Cytometry

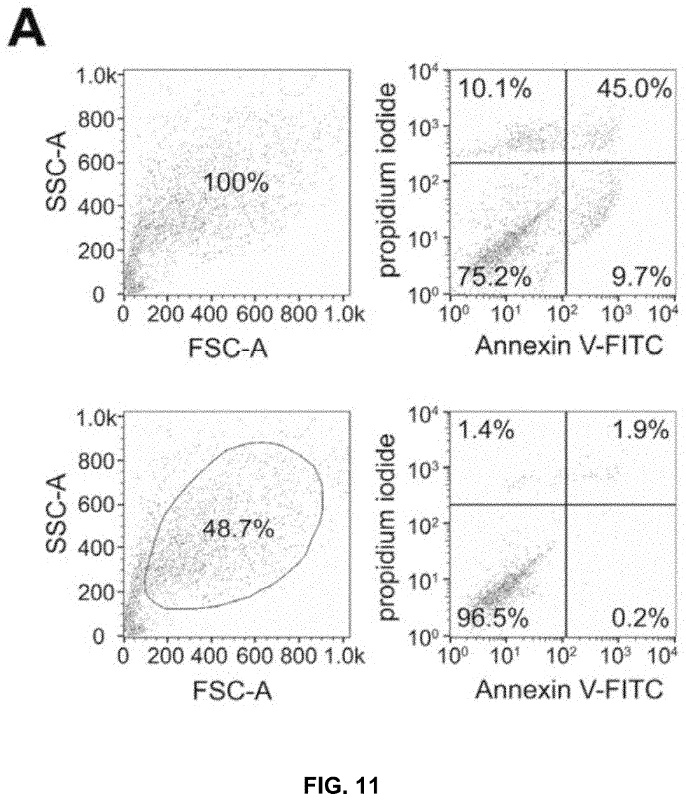

The naïve hDMCs were evaluated to be alive at a rate of 75.2%, as measured by the Annexin V-fluorescein isothiocyanate/propidium iodide apoptosis assay. Following cell gating, a population composed of more than 95% live cells was selected for integrin analysis (FIG. 11). This cell gating was also applied for other analyses. In the population selected through cell gating, expressions of integrin subtypes were evaluated.

6-2. Assay for Integrin Subtype Expression in Naïve hDMCs by Flow Cytometry

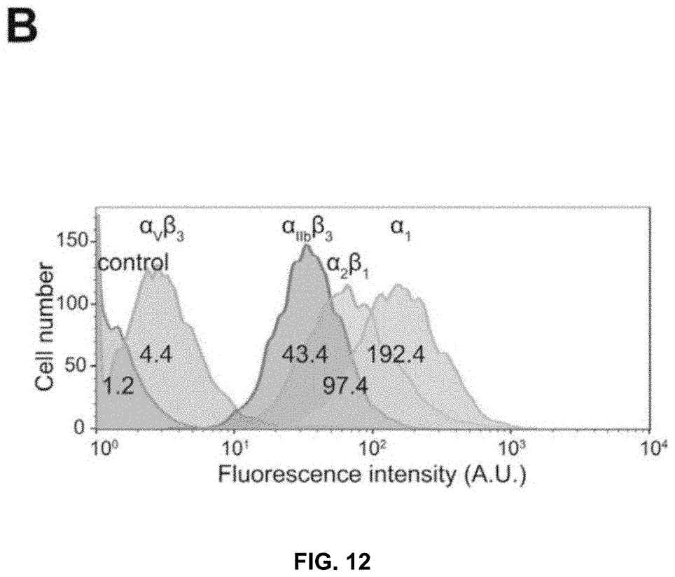

The direct immunofluorescence labeling and FACS results showed that integrins α1, α2β1, and αIIbβ3 were expressed at higher levels than αVβ3. Expressing cells were counted to be 192.4±149.9 for integrin α1, 97.4±177.6 for integrin α2β1, 43.4±154.2 for integrin αIIbβ3, and 4.4±10.0 for integrin αVβ3 (FIG. 12).

From the results, it was understood that naïve hDMCs expressed integrins α1 and α2β1 at higher levels than integrin αIIbβ3 and αVβ3.

6-3. Assay for Relationship Between Inflammation and Integrin Subtype Expression by Flow Cytometry

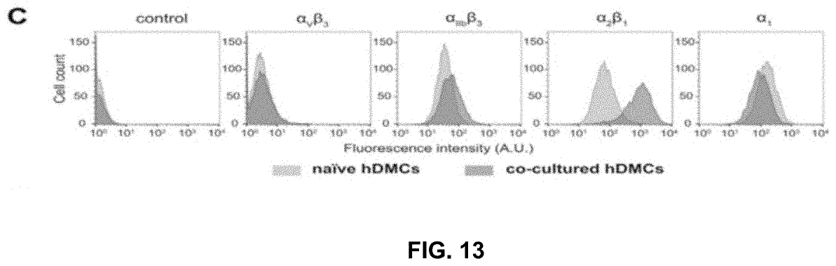

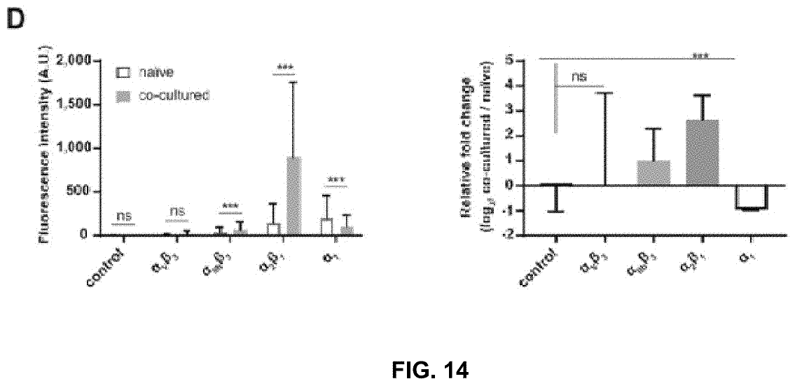

To determine the relationship between inflammation and integrin expression, the expression of integrins was compared between MΦ-co-cultured and naïve cells (FIG. 13). Compared with naïve cells, the co-cultured cells increased the expression of integrin α2β1 by 6.3-fold (142.7±222.8 for naïve and 898.8±859.1 for co-cultured; p<0.001) and αIIbβ3 by 2-fold (32.7±64.7 for naïve and 65.8±94.8 for co-cultured; p<0.001) (FIG. 14), but decreased the expression of integrin α1 by 2-fold (196.9±261.8 for naïve and 100.2±135.1 for co-cultured; p<0.001). There were almost no differences in the expression level of integrin αVβ3 therebetween (4.1±11.4 for naïve and 4.1±49.9 for co-cultured).

The co-cultured cells in inflammation exhibited an increased expression level of integrin α2β1 among others, compared with naïve cells, and integrin α2β1 was understood to play a critical role in cell adhesion.

### Example 7

### Functional Validation of Integrin α2β1 by Treatment with Intedrin α2β1 Inhibitor

For the functional validation of integrin α2β1 in the adhesion of hDMCs, cell viability and adhesion assays of hDMCs were performed with treatment of the selective integrin α2β1 inhibitor BTT 3033.

7-1. Cytotoxicity Assay of BTT 3033

For cytotoxicity assay of BTT 3033, hDMCs were treated with BTT 3033 in a dose dependent manner and incubated at 37° C. for 90 min. After incubation, cell viability assay was performed.

TABLE 4

| BTT 3033 concentration (nM) | Viability (A.U.) |
|---|---|
| 0 (control) | 1.000 ± 0.090 |
| 16.25 | 0.896 ± 0.090 |
| 32.50 | 0.844 ± 0.099 |
| 65.00 | 0.899 ± 0.075 |
| 130.00 | 0.800 ± 0.054 |
| 260.00 | 0.854 ± 0.081 |
| 520.00 | 0.827 ± 0.069 |

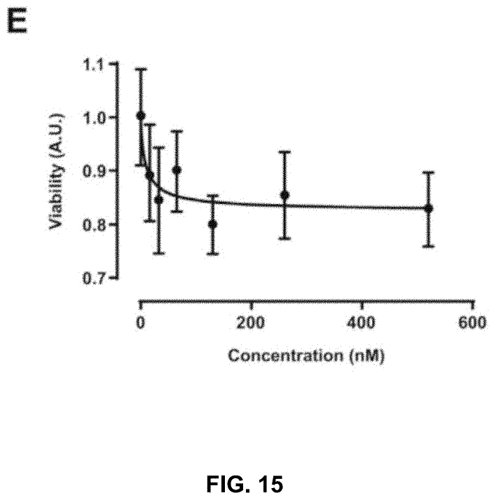

The cytotoxicity of BTT 3033 was found to be less than 20.0% at a concentration from 16.25 to 520.00 nM (FIG. 15, Table 4). Since BTT 3033 has a half maximal effective concentration (EC₅₀) at 130 nM according to manufacturer's instructions, 130 nM was determined as the therapeutic dose.

7-2. Functional Validation of Integrin a2β1 by Treatment with Integrin α2β1 Inhibitor

For an adhesion assay for the functional validation of integrin α2β1, the co-cultured hDMCs were incubated with 130 nM BTT 3033 at 37° C. for 30 min. After incubation, an adhesion assay was performed as described above.

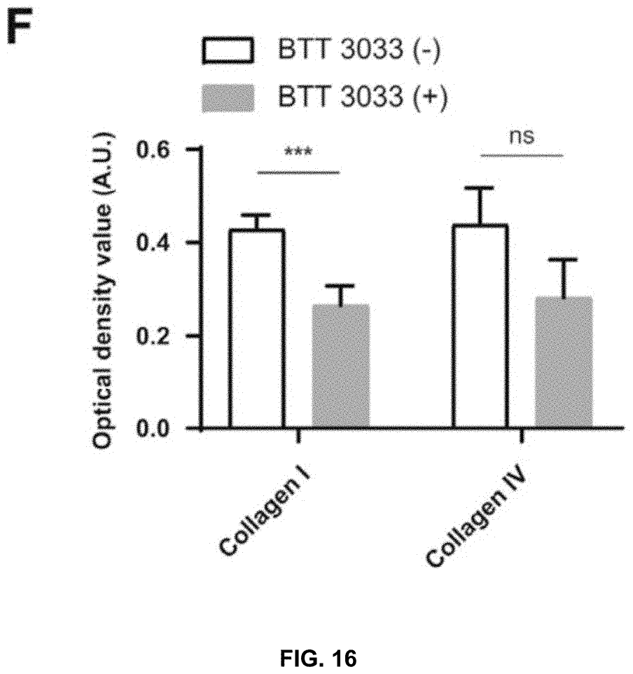

The treatment with BTT3033 reduced the adhesion of the co-cultured hDMCs to collagen I by 37.8% (0.43±0.03 for un-treated and 0.27±0.04 for BTT 3033-treated; p<0.001) and to collagen IV by 35.7% (0.44±0.08 for un-treated and 0.28±0.08 for BTT 3033-treated; p=0.057) (FIG. 16).

The data shows the reduced adhesion of hDMCs to the ECM components collagen I and IV by treatment with the integrin α2β1 inhibitor BTT 3033, suggesting that the inhibition of integrin α2β1 leads to a reduction in the adhesion of hDMCs in inflammation.

### Example 8

### ELISA Assay for Levels of MMPs, TIMPs and VEGF in hDMCs

MMP1 mainly interacts with collagens I, II, III, VII, and X, and has an influence on tissue regeneration as well as tissue degradation. MMP3 interacts with collagens IV, V, IX, and X, laminin, and fibronectin, acting as a mediator of inflammation-mediated tissue degradation. TIMP is known to be an endogenous inhibitor of MMPs and a regulator of cell proliferation and apoptosis. In addition, VEGF is a factor responsible for angiogenesis associated with wound healing.

Levels of Matrix metalloproteinases (MMPs), tissue inhibitors of metalloproteinases (TIMPs), and vascular endothelial growth factor (VEGF) in naïve hDMCs and MΦ-co-cultured hDMCs were measured by ELISA.

Media from naïve hDMC cultures, MΦ cultures, and co-cultures of MΦ and hDMCs were assayed. Levels of MMP-1, MMP-3, MMP-9, MMP-2, TIMP-1, TIMP-2, and VEGF were assayed using DuoSet ELISA Development System kits (MMP-1: DY901, MMP-3: DY513, MMP-9: DY911, MMP-2: DY902, TIMP-1: DY970, TIMP-2: DY971, and VEGF: DY293B; R&D Systems).

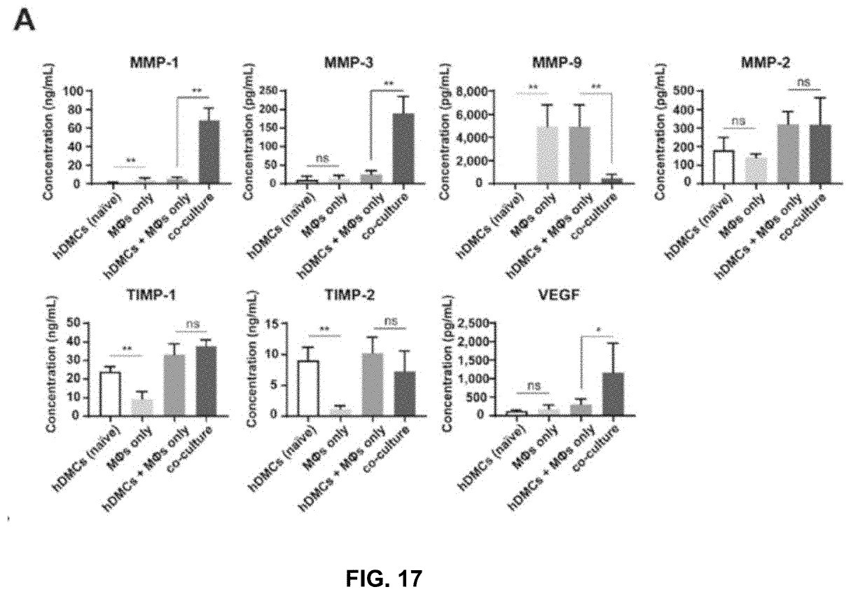

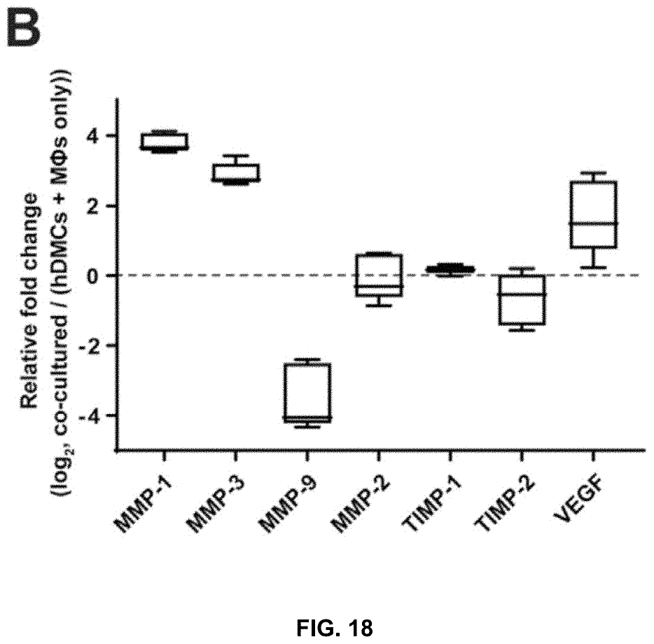

Naïve hDMCs were measured to be lower in MMP-1 and MMP-9 levels, but higher in TIMP-1 and TIMP-2 levels than MΦ (p<0.01). However, there were no statistical differences between the basal levels of MMP-3, MMP-2, or VEGF therebetween (FIG. 17, Table 5). To investigate changes in MMPs, TIMPs, and VEGF during inflammation, comparison was made of the levels among the co-cultured cells, the naïve hDMC, and the MΦ (FIG. 18). Co-culturing of hDMCs with MΦ induced significant increases in MMP-1, MMP-3, and VEGF expression compared with naïve hDMCs and MΦ alone (MMP-1: 13.9-fold change, p<0.01; MMP-3: 7.6-fold change, p<0.01; VEGF: 3.8-fold change, p<0.05). In contrast, there were no changes in MMP-2, TIMP-1, and TIMP-2 expression between groups, and the expression of MMP-9 appeared to decrease.

Taken together, the results show that MMP-1 and MMP-3, together with integrin α2β1, play a critical role in hDMC adhesion.

In the co-cultured cells, no changes in TIMP-1 were detected whereas increased levels of MMP-1 and MMP-3 were measured. Such experiment results implicate that the change of MMP is not attributed to TIMP, but driven by the interaction between hDMCs and MΦ.

Moreover, a wound healing process is understood to progress through the interaction between hDMCs and MΦ when considering the increase level of VEGF.

TABLE 5

| | hDMCs | | | Co-cultured | Fold |
|---|---|---|---|---|---|
| | (naïve) | Mϕ | hDMCs + Mϕ | hDMCs | change |
| MMP-1 (ng/ml) | 1.1 ± 0.8 | 3.8 ± 2.5 | 4.9 ± 2.3 | 68.6 ± 13.1 | 13.9 ± 2.6 |
| MMP-3 (pg/ml) | 11.1 ± 9.1 | 14.1 ± 8.8 | 25.2 ± 10.0 | 190.5 ± 45.1 | 7.6 ± 1.8 |
| MMP-9 (pg/ml) | 0 | 4,933.3 ± 1,887.6 | 4,933.3 ± 1,887.6 | 481.9 ± 320.7 | 0.1 ± 0.1 |
| MMP-2 (pg/ml) | 180.5 ± 69.0 | 141.2 ± 18.2 | 321.7 ± 67.9 | 319.5 ± 144.2 | 1.0 ± 0.4 |
| TIMP-1 (ng/ml) | 23.9 ± 2.7 | 9.3 ± 3.8 | 33.3 ± 5.7 | 37.7 ± 3.46 | 1.1 ± 0.1 |
| TIMP-2 (ng/ml) | 9.1 ± 2.1 | 1.2 ± 0.5 | 10.2 ± 2.6 | 7.2 ± 3.3 | 0.7 ± 0.3 |
| VEGF (pg/ml) | 125.8 ± 30.0 | 182.9 ± 108.2 | 308.7 ± 136.5 | 1,163.4 ± 795.3 | 3.8 ± 2.6 |

### Example 9

### qRT-PCR Assay for Levels of MMPs, TIMPs and VEGF in hDMCs

RNA was isolated from the naïve hDMCs, the MΦ, and the co-cultured hDMCs, using RNeasy Mini Kit (QIAGEN, Valencia, Calif.). Concentration and purity of RNA were determined using Nanodrop 2000 (Thermo Fisher Scientific, Waltham, Mass., USA). The complementary DNA was synthesized from the RNA with the aid of a Maxime RT PreMix Kit (iNtRON Biotechnology, Gyeonggi-do, Korea). The primers for GAPDH, MMP-1, and MMP-3 were used (Table 6).

TABLE 6

| | | | SEQ |
|---|---|---|---|
| | | | ID |
| Gene | Direction | Sequence | NO: |
| GAPDH | sense | GTGAACCATGAGAAGTATGACAA | 1 |
| | antisense | CATGAGTCCTTCCACGATAC | 2 |
| MMP-1 | sense | CTGAAGGTGATGAAGCAGCC | 3 |
| | antisense | AGTCCAAGAGAATGGCCGAG | 4 |
| MMP-3 | sense | CTCACAGACCTGACTCGGTT | 5 |
| | antisense | CACGCCTGAAGGAAGAGATG | 6 |

To determine which cells of hDMCs or MΦ induced such statistical changes, qRT-PCR analysis was performed. Using 2^{-ΔΔCt} method, mRNA expression fold change ratios between the individual cell groups and the co-cultured cells were calculated for each of MMP-1 (1.20±1.76 for MΦ and 4.58±2.79 for hDMCs) and MMP3 (68.27±9.28 for MΦ and 1.02±0.96 for hDMCs) (FIG. 17).

From the results, it was found that the increased expression of MMP-1 was affected by both hDMCs and MΦ whereas the increased expression of MMP-3 was attributed mainly to MΦ. In addition, the expression of MMP-9 was measured to decrease. Thus, the data imply that the expression of MMP-9 is downregulated in hDMCs, whereby the adhesion of hDMCs is affected.

Discussion

hDMC adhesion to ECM is crucial for determining the prognosis after treatment of spinal disease. In this disclosure, hDMC adhesion was examined through experiments.

In the disclosure, it was found that collagen I and IV are critical components responsible for hDMC adhesion and that pathological conditions related to dura mater inflammation are influenced by increases in integrin subtype a2b1, MMP-1, and MMP3, and decreases in integrin α1 and MMP-9.

These findings may be helpful for the treatment of diseases related to dura mater adhesion. Particularly, integrin α2β1 can be a therapeutic target for hDMC adhesion and fibrosis in inflammatory conditions and the inhibition of integrin α2β1 will lead to a therapeutic effect thereon.

What is claimed is:

1. A method for prevention or treatment of tissue adhesion, the method comprising a step of administering a pharmaceutical composition comprising an integrin α2β1 inhibitor as an active ingredient to a subject.

2. The method of claim 1, wherein the integrin α2β1 inhibitor is selected from the group consisting of an antibody, an aptamer, a natural extract, and a compound.

3. The method of claim 1, wherein the integrin α2β1 inhibitor is selected from the group consisting of an anti-integrin α2β1 antibody or an antigen-binding fragment thereof, BTT 3016, BTT 3033, BTT 3034, and rhodocetin.

4. The method of claim 1, wherein the tissue adhesion is selected from the group consisting of peritoneal adhesion, pelvic adhesion, pericardial adhesion, peritendinous adhesion, and peridural adhesion.

5. The method of claim 1, wherein the pharmaceutical composition is in a formulation form selected from the group consisting of a medicine for internal use, an injection, a gel, a film, a perfusate, a spray liquid, an atomizing or vaporizing liquid, a foaming aerosol, and an infusion.

6. A method for screening a material for prevention or treatment of tissue adhesion, the method comprising the steps of:
(a) co-culturing macrophage-like THP-1 cells with collagen I-expressing cells or cells transformed to express collagen I;
(b) treating the co-cultured collagen I-expressing cells with a candidate material;
(c) measuring an expression level of an integrin α2β1 protein or gene in the collagen I-expressing cell treated with the candidate material; and
(d) determining the candidate material to be a material for prevention or treatment of tissue adhesion when the measured expression level of the integrin α2β1 protein or gene in step (c) is reduced, compared to untreated, collagen I-expressing cells.

7. The method of claim 6, wherein the tissue adhesion is selected from the group consisting of peritoneal adhesion, pelvic adhesion, pericardial adhesion, peritendinous adhesion, and peridural adhesion.

8. An anti-adhesion composition comprising an integrin α2β1 inhibitor and a biocompatible hydrogel.

9. The anti-adhesion composition of claim 8, wherein the integrin α2β1 inhibitor is selected from the group consisting of an antibody, an aptamer, a natural extract, and a compound.

10. The anti-adhesion composition of claim 8, wherein the integrin α2β1 inhibitor is selected from the group consisting of an anti-integrin α2β1 antibody or an antigen-binding fragment thereof, BTT 3016, BTT 3033, BTT 3034, and rhodocetin.

11. The anti-adhesion composition of claim 8, wherein the adhesion is selected from the group consisting of peritoneal adhesion, pelvic adhesion, pericardial adhesion, peritendinous adhesion, and peridural adhesion.

12. The anti-adhesion composition of claim 8, being in a form of a gel or a film.
